(19) United States
(12) Patent Application Publication
(10) Pub. No.: US 2024/0055108 A1
(43) Pub. Date: Feb. 15, 2024

(54) Method for Assigning a Medical Device

(71) Applicant: Siemens Healthcare GmbH, Erlangen (DE)

(72) Inventors: Mathias Nittka, Baiersdorf (DE); Armin Stranjak, Uttenreuth (DE); Christoph Forman, Uttenreuth (DE); Robert Grimm, Nürnberg (DE)

(73) Assignee: Siemens Healthcare GmbH, Erlangen (DE)

(21) Appl. No.: 18/233,392

(22) Filed: Aug. 14, 2023

(30) Foreign Application Priority Data
Aug. 15, 2022 (EP) 22190373.5

(51) Int. Cl. G16H 40/20

(52) U.S. Cl. CPC G16H 40/20; G16H 10/60

(57) ABSTRACT

A method for assigning a medical device may be implemented in a scheduler agent apparatus. The method may include receiving, from a patient agent apparatus, a utilization request containing patient parameters for examining a patient by a medical device from the group; determining the suitability of at least one medical device from the group for the examination based on the patient parameter or parameters and one or more device parameters; assigning a medical device determined to be suitable to the utilization request based on the patient parameter or parameters and device parameters; transmitting, to the patient agent apparatus, a notification regarding the assignment; and transmitting a notification to an device agent apparatus associated with the assigned medical device.

### CROSS REFERENCE TO RELATED APPLICATIONS

This patent application claims priority to European Patent Application No. 22190373.5, filed Aug. 15, 2022, which is incorporated herein by reference in its entirety.

### BACKGROUND

### Field

The present disclosure relates to a computer-implemented method for assigning a medical device to an examination of a patient, wherein the medical device is assigned from a group of, in particular similar, medical devices. The disclosure furthermore relates to a scheduler agent apparatus, a device agent apparatus, a system comprising the scheduler agent apparatus, a plurality of device agent apparatuses, one or more patient agent apparatuses, a computer program and a computer-readable storage medium.

### Related Art

Conventionally, larger hospital radiological departments or generally healthcare providers operate fleets of imaging medical devices, for example magnetic resonance tomography (MRT) scanners. The MRT scanners in the fleet are not usually identical, but can include a plurality of different hardware and software versions, such as, for example, different field strengths and performance specifications, different imaging modalities, such as neurological, vascular or orthopedic imaging applications with associated special equipment, such as, for example, coils and software packages. The MRT scanners or MRT systems can, for example, be specialized for routine imaging, clinical research or special purposes such as emergencies, intraoperative interventions or for scheduling radiation treatment.

The question as to which patient should be directed to which scanner is in no way a trivial task: depending upon the imaging required by the client for a specific diagnostic question, the scanner selected must offer the appropriate imaging options (for example hardware, licenses, installed software including sequences, reconstruction algorithms and/or protocols). A further factor is internal and external guidelines, such as, for example, quality standards and/or compatibility requirements. Further factors to consider include the scanner location (for example whether the scanner needs to be in the same building or can be located more remotely) and availability in terms of utilization or operating hours.

Therefore, the task of scheduling a patient for a specific scanner conventionally requires highly qualified staff, the availability of very complex information about the scanner's infrastructure and a large amount of time for acquiring the necessary information. Conventionally, this can lead to suboptimal patient treatment, suboptimal scanner utilization, time-consuming scheduling processes and/or scheduling errors and subsequently to safety-critical situations.

Conventional patient scheduling is further based on institutional rules that are generally simplifications, so that scanner utilization is not optimal.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 shows a flowchart of a method according to an exemplary embodiment of the disclosure.

FIG. 2 shows a block diagram of a scheduler agent apparatus according to an exemplary embodiment of the disclosure, where the scheduler agent apparatus may be configured to execute the method according to FIG. 1.

FIG. 3 shows a block diagram of a device agent apparatus according to an exemplary embodiment of the disclosure.

FIG. 4 shows a schematic example of a system comprising a scheduler agent apparatus, for example according to FIG. 2, a plurality of device agent apparatuses, for example according to FIG. 3, and a plurality of patient agent apparatuses, according to exemplary embodiments of the disclosure.

FIG. 5 shows a method according to an exemplary embodiment of the disclosure with assignment of the steps to the respective agent apparatuses.

FIG. 6 shows a method according to an exemplary embodiment of the disclosure with assignment of the steps to the respective agent apparatuses.

FIG. 7 shows an example of an assignment of medical devices to patients depending on a time of the scheduled examination, according to an exemplary embodiment of the disclosure.

FIG. 8 shows the optimization of the assignment between a medical device and a patient in dependence on the time of the scheduled examination according to an exemplary embodiment of the disclosure.

FIG. 9 shows the optimizing the assignment between medical devices and examinations of patients according to an exemplary embodiment of the disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

The present disclosure is based on the object of enabling automated assignment of (in particular imaging) medical devices to (for example examinations of) patients that is optimized both with regard to the utilization of a plurality of (in particular imaging) medical devices with regard to patient-specific requirements, in particular with regard to technical equipment and/or (in particular image) quality of the medical device and with regard to the urgency of the examination of (and/or use of the medical device by) the patient and/or with regard to patient mobility.

According to one method aspect, a computer-implemented method is provided for assigning a (for example imaging) medical device from a group of medical devices to an examination of a patient. The method is implemented in a scheduler agent apparatus. The method comprises a step of receiving, from a patient agent apparatus, a utilization request for examining a patient by means of a medical device from the group of medical devices. The utilization request comprises at least one patient parameter of the patient. The method further comprises a step of determining the suitability of at least one medical device from the group of medical devices for the examination based on the at least one received patient parameter and at least one device parameter of the medical device. The method further comprises a step of assigning one of the at least one medical device determined to be suitable to the received utilization request for the examination of the patient based on the at least one received patient parameter and the at least one device parameter. The method further comprises a step of transmitting a notification to the patient agent apparatus. The notification comprises the assigned medical device determined to be suitable for the examination of the patient. The method further comprises a step of transmitting a further notification to a device agent apparatus associated with the assigned medical device. The further notification comprises the assigned examination of the patient.

The assignment between the medical device and the patient can also be referred to as allocation (for example of the patient to the medical device, or vice versa).

The medical device, or at least one or each medical device from the group of medical devices, can comprise an imaging device for executing an imaging method, for example a device for magnetic resonance tomography (MRT), computed tomography (CT), X-ray imaging, ultrasound imaging, and/or positron emission tomography (PET), in particular for single photon emission computer tomography (SPECT for short). In particular depending on the context, each abbreviation for an imaging method can correspond to an abbreviation for the corresponding device. For example, MRT can denote both the method for magnetic resonance tomography and the device for executing the method—a magnetic resonance tomography unit.

The medical device can be embodied to combine two or more imaging methods, for example for a combination of PET and CT and/or a combination of PET and MRT.

The group of medical devices can also be referred to as a fleet of medical devices (also: fleet for short).

The group of medical devices can comprise a plurality of medical devices, in particular of a predetermined type (wherein the type of medical device can also be referred to as a device type) at a location and/or in a predetermined area. The location can, for example, comprise a medical facility, in particular a hospital, a hospital campus and/or a medical center.

Alternatively, or additionally, the predetermined area can, for example, comprise a city, a district and/or an area of an organizational association of multiple medical facilities.

The plurality of medical devices in the group of medical devices can, for example, comprise from two to fifty medical devices at one location and/or up to several hundred medical devices in a predetermined area, for example in a major city.

The examination of the patient can comprise the execution of an imaging method by means of the appropriate imaging device.

The at least one patient parameter of the patient can, for example, comprise the height, weight, age, body part to be examine and/or presence of an implant. Alternatively, or additionally, the patient parameters could also include medical diagnoses, such as, for example, suspected fracture of a vertebral body and/or the appropriate preferred diagnostic procedures, such as, for example, imaging (for example MRT) of the spine.

The at least one device parameter can, for example, comprise magnetic field strength and/or the presence of antenna coils for body parts to be examined for an MRT and/or a number of X-ray sources of a CT.

The step of assigning can be preceded by a step of transmitting an availability request and/or receiving availability information.

The steps of transmitting the notification to the patient agent apparatus and of transmitting the further notification to the device agent apparatus can be executed independently of one another and/or in any sequence.

The step of assigning can comprise optimization.

The computer-implemented method can be used to optimize utilization and/or uniform usage of the medical devices in the group of medical devices. Alternatively, or additionally, the technical suitability of the medical device for the examination of the patient can be optimized. Optimized technical suitability can enable improved diagnosis and/or an improved outcome of the examination of the patient. Further alternatively or additionally, the method can optimize a time window for the examination of the patient, for example the examination can be performed at the earliest possible time at which one of the devices from the group of medical devices is available and suitable for the examination.

The computer-implemented method can enable, in particular optimized, assignment of the examination of the patient to a medical device within a short period of time, for example within a few minutes.

The group of medical devices can comprise a type of medical devices for a predetermined type of patient examinations.

The medical device can comprise an imaging device. Alternatively, or additionally, the group of medical devices can comprise a group of imaging devices.

The imaging device, and/or the group of imaging devices, can comprise a magnetic resonance tomography unit (MRT). Alternatively, or additionally, the imaging device, and/or the group of imaging devices, can comprise a computed tomography unit (CT). Alternatively, or additionally, the imaging device, and/or the group of imaging devices, can comprise an X-ray device. Alternatively, or additionally, the imaging device, and/or the group of imaging devices, can comprise an ultrasound device. Alternatively, or additionally, the imaging device, and/or the group of imaging devices, can comprise a positron emission tomography unit (PET), in particular a single photon emission computed tomography unit (SPECT). Further alternatively or additionally, the imaging device, and/or the group of imaging devices, can comprise a combination of two or more imaging devices, in particular a combination of PET and CT, and/or a combination of PET and MRT.

The method may further comprise a step of transmitting an availability request to at least one of the at least one medical device determined to be suitable. The availability request can be transmitted to the device agent apparatus associated with the respective medical device determined to be suitable.

Alternatively, or additionally, the method can comprise a step of receiving availability information for the at least one medical device determined to be suitable, for example in response to the availability request. The availability information can be received from the device agent apparatus associated with the respective medical device determined to be suitable.

The transmission of the availability request can comprise the question as to whether the medical device is ready for operation and/or as to whether the medical device is already scheduled elsewhere and/or is being used for another examination on another patient.

The availability information can be received in response to the availability request. Alternatively, or additionally, the availability information can comprise availability information initiated by the device agent apparatus.

The availability information initiated by the device agent apparatus can comprise periodic information, for example a periodic “ping” to notify operational readiness of the medical device. Alternatively, or additionally, the availability information initiated by the device agent apparatus can comprise event-oriented information. The event-oriented information can comprise an idle state and/or a free availability of the medical device. Alternatively, or additionally, the event-oriented information can comprise an error message and/or a message notifying non-availability.

Alternatively, or additionally to the steps of transmitting an availability request and/or of receiving availability information, the method can comprise the use of a watchdog timer. A watchdog timer can be embodied to check whether availability information for the medical device is received in predetermined time windows and/or at predetermined time intervals.

Non-receipt and/or absence of availability information, in particular after the expiry of the predetermined time window and/or the predetermined time interval can display or indicate a fault scenario and/or non-availability of the medical device.

A detected change to the availability of a medical device can be used as the basis for changing a previously determined assignment. Alternatively, or additionally, the steps of receiving the utilization request and/or determining the suitability of the medical device (and/or the at least one device parameter) can be executed once, while subsequent steps relating to the assignment between patients and medical devices can be executed multiple times, for example due to a change in the availability of at least one medical device from the group of medical devices.

The at least one patient parameter can comprise a medical question. Alternatively, or additionally, the at least one patient parameter can comprise part of the patient's body to be examined. Alternatively, or additionally, the at least one patient parameter can comprise a patient's physique, in particular a height and/or a weight. Alternatively, or additionally, the at least one patient parameter can comprise the presence of an implant at or near the part of the patient's body to be examined (for example a pacemaker in the case of examinations of the torso and/or chest region). Alternatively, or additionally, the at least one patient parameter can comprise a patient's age. Alternatively, or additionally, the at least one patient parameter can comprise a patient's gender. Alternatively, or additionally, the at least one patient parameter can comprise a patient's mobility. Alternatively, or additionally, the at least one patient parameter can comprise a patient's mental fitness. Alternatively, or additionally, the at least one patient parameter can comprise a patient's physical fitness. Alternatively, or additionally, the at least one patient parameter can comprise the urgency of the examination of the patient. Alternatively, or additionally, the at least one patient parameter can comprise a requirement for a device parameter, for example a requirement for a magnetic field strength of an MRT. Further alternatively or additionally, the at least one patient parameter can comprise participation in a clinical trial.

The at least one patient parameter can comprise two or more patient parameters, for example a combination of the body part to be examined, mobility and the urgency of the examination of the patient and/or the medical question, for example a question about a torn ligament by a referring physician or another medical facility. Alternatively, or additionally, the body part to be examined can be expressed in more concrete terms by the medical question. For example, the body part to be examined can comprise a knee joint and the medical question whether or not there is a torn ligament at the knee joint. In this case, the medical question or diagnosis can in particular also be represented as “Diagnostic Related Groups” code (DRG code for short, for example retrievable via https://www.icd-code.de/icd/code/ICD-10-GM.html), “International Classification of Diseases” code (ICD-10 code for short, for example retrievable via https://www.icd-code.de/icd/code/ICD-10-GM.html) and/or a “Current Procedural Terminology” code (CPT code, for example retrievable via https://www.cms.gov/license/ama?file=/files/zip/list-codes-effective-j anuary-1-2022-published-november-19-2021.zip). For example, the ICD-10 code M99.23 classifies sublexation stenosis of the spinal channel, lumbar region [lumbosacral], as a biomechanical lesion. Alternatively, or additionally, the CPT code 72159 stands for “MR angio spine w/o&w/dye” (“MR angiography spine without contrast medium followed by with contrast medium”).

The body part to be examined can comprise the patient's head, neck, spine, abdomen, and/or one or more of the patient's joints (for example a knee joint). Alternatively, or additionally, the body part to be examined can, for example, be classified as neurological, related to one or more vessels (for example at least one blood vessel and/or one or more vessels), and/or orthopedic. Further alternatively or additionally, one or more preferred diagnostic procedures can, for example, be defined by specifying the corresponding CPT codes.

The physique can comprise the patient's weight, for example normal weight, underweight and/or overweight. Alternatively, or additionally, the physique can comprise the patient's height and/or width.

The patient's physique can limit or prevent the assignment of a medical device comprising a tube and/or a tunnel.

The implant can contain metal. The presence of a metal-containing implant can limit or prevent the assignment of a medical device.

The implant can, for example, comprise a pacemaker, a stent, an artificial joint (for example an artificial knee joint and/or an artificial hip joint) and/or a dental implant.

The patient's age can limit an assignment of the medical device (for example by medical guidelines and/or legal requirements). For example, the maximum X-ray dose may be limited for a child.

The patient's mobility can comprise the patient's physical fitness and/or state of health. For example, it may not be possible for a patient who is to be examined with regard to possible injuries following an accident to be transported between different medical locations.

The patient's mental fitness can comprise claustrophobia (also: fear of confined or enclosed spaces). Alternatively, or additionally, the patient's mental fitness can comprise calmness, nervousness and/or self-control.

The patient's physical fitness can, for example, comprise the ability to hold the breath for a predetermined period (for example up to 15 seconds or up to a few minutes).

The urgency of the examination can, for example, comprise a maximum time frame for scheduling the examination. For example, the urgency of an examination of the patient after an accident and/or in an emergency can comprise an immediate examination or an examination in up to a maximum of several hours (for example two hours). Alternatively, or additionally, no follow-up examination can be classified as non-urgent, for example, the examination can be scheduled several days or several weeks in advance.

Further alternatively or additionally, the urgency of the examination of the patient can comprise an intra-operative examination by means of the medical device. The urgency of the examination can correspond to the scheduling of the operation.

The requirement for a device parameter can comprise a requirement for image contrast, a type of imaging, for example comprising a contrast medium, an (in particular magnetic) field strength, and/or an X-ray dose.

The requirement for the X-ray dose can comprise or be limited by an X-ray dose already received by the patient over a predetermined prior period of time. Alternatively, or additionally, the X-ray dose can be determined by a recommendation, for example depending on age and/or gender.

Participation in a clinical trial can result in a restriction of the device parameters and/or the assignment of the examination of the patient to a predetermined subgroup of the group of medical devices.

The at least one device parameter can comprise a location of the medical device. Alternatively, or additionally, the at least one device parameter can comprise a design of the medical device, in particular comprising a tube, semi-open and/or framework-based. Alternatively, or additionally, the at least one device parameter can comprise the loading capacity and/or mobility of a patient bench. Alternatively, or additionally, the at least one device parameter can comprise the presence of special accessories (for example antenna coils for MRT). Alternatively, or additionally, the at least one device parameter can comprise the field strength, in particular magnetic field strength. Alternatively, or additionally, the at least one device parameter can comprise a number of X-ray sources and associated detectors. Alternatively, or additionally, the at least one device parameter can comprise the image quality. Alternatively, or additionally, the at least one device parameter can comprise the presence of special software for performing and/or evaluating the examination. Alternatively, or additionally, the at least one device parameter can comprise the average duration of a type of examination corresponding to the utilization request. Alternatively, or additionally, the at least one device parameter can comprise the time availability. Alternatively, or additionally, the at least one device parameter can comprise the presence of trained personnel. Further alternatively or additionally, the at least one device parameter can comprise suitability for clinical trials.

The at least one device parameter can comprise two or more device parameters. For example, the at least one device parameter can comprise a location, magnetic field strength and the presence of antenna coils for MRT.

The location of the medical device can comprise the location of a medical facility, building, floor and/or a type of room, for example an operating theater.

The design of the medical device can comprise a tube and/or a tunnel. Alternatively, or additionally, the at least one device parameter can comprise a tunnel diameter and/or a tube diameter.

Alternatively, or additionally, the design of the medical device can be semi-open and/or comprise a gantry, in particular a sliding gantry.

The loading capacity of the patient bench can comprise a predetermined maximum weight of the patient, including, for example, any special accessories to be attached to the patient.

Alternatively, or additionally, the mobility of the patient bench can comprise whether the patient bench can be partially or completely moved into a tube or a tunnel or whether it is possible to move a patient on the bench from a preparation room without that being necessary to transfer the patient to the device's patient bench.

The special accessories can also be referred to as hardware equipment. The special accessories can comprise a contrast medium injection facility, an optical stimulator, a facility for measuring heartbeat or heart current (for example an electrocardiogram, ECG, sensor), an organ puncture facility, a tumor biopsy facility, a ventilation facility, a sedation facility and/or a signal receiving site for a patient body part. The signal receiving site can comprise a local coil (also: antenna coil) for MRT.

The contrast medium injection facility can be used for the visualization of blood vessels, in particular arteries, with the aid of a contrast medium. Visualization by contrast medium can also be referred to as “angiography” (or also “arteriography”). Alternatively, or additionally, in angiography, contrast medium can be injected directly into a blood vessel, in particular an artery, while simultaneously an X-ray image is taken in order to visualize the blood vessel, in particular the artery, in the X-ray image.

The organ puncture facility can, for example, be used with an ultrasound device as a medical device. If, for example, an ultrasound examination (also: sonography), CT, and/or MRT reveals suspicious structures in a patient's organ, for example the liver, pancreas and/or lymph nodes, these structures can be selectively punctured under ultrasound control. For example, this in particular enables a tissue sample to be taken from the organ for histological examination.

The field strength can comprise an MRT magnetic field strength. The MRT magnetic field strength can, for example, be 0.5 tesla (T for short); 0.55 T; 1.5 T; 3 T; 7 T and/or 9.4 T. A field strength of 0.5 T can be referred to as a low field strength. Alternatively, or additionally, field strengths of at least 7 T can be referred to as field strengths for research purposes.

In particular for CT, the number of X-ray sources and associated detectors can be one or two. A CT with one X-ray source and the associated detector can also be referred to as a “single-source CT”. A CT with two X-ray sources and associated detectors can also be referred to as a “dual-source CT”. Alternatively, or additionally, a (for example average) duration of an examination with two X-ray sources can be shorter than a corresponding examination with one X-ray source. A shorter examination time can in particular be advantageous for trauma patients and/or patients being examined as the result of an accident. For example, a traffic accident and/or a fall can be referred to as trauma.

Image quality, in particular spatial resolution, can increase with the value of the field strength, in particular the magnetic field strength of an MRT.

The image quality can comprise spatial resolution and/or temporal resolution. Conventional spatial resolution can be in the range of 0.3 mm to 1.5 mm, for example with MRT. Alternatively, or additionally, at a higher field strength and/or for research purposes, spatial resolution can be in the sub-mm range and/or less than millimeter (mm).

Alternatively, or additionally, the image quality can comprise image noise (in particular low image noise).

Image noise can be defined as the deterioration of a digital or electronically recorded image due to interference that has no relation to the actual image content and/or the image signal. Interfering pixels can, for example, deviate from those of the actual image in terms of color and/or brightness.

Special software can comprise an examination protocol and/or a measurement protocol. An examination protocol can in particular enable an examination in the presence of an implant as a patient parameter for example.

Alternatively, or additionally, special software can comprise one or more sequences, in particular measurement sequences. A measurement sequence can, for example, comprise a number and/or width of (for example electromagnetic) pulses, a magnetic field connection and/or turbo spin echo (TSE). Alternatively, or additionally, the measurement sequence can comprise spin echo (SE), spin echo fat saturation (SE fs;), fluid attenuated inversion recovery (FLAIR) and/or gradient echo (GRE). Further alternatively or additionally, the measurement sequence can comprise diffusion weighted imaging (DWI) and/or perfusion weighted imaging (PWI), in particular for stroke diagnosis. Furthermore, alternatively, or, the measurement sequence can comprise turbo inversion recovery magnitude (TIRM), short tau inversion recovery (STIR) and/or diffusion tensor imaging (DTI).

Further alternatively or additionally, special software can be suitable for performing functional MRT (fMRT for short; or fMRI for functional magnetic resonance imaging), for example for a functional examination of the brain. fMRT can be used to visualize physiological functions inside the body using MRT methods.

The average duration of a type of examination can correspond to an empirical value and/or be used for time scheduling by the scheduler agent apparatus.

The time availability of the medical device can comprise availability according to the (for example existing) appointment schedule. The appointment schedule can also be referred to as a time schedule.

Alternatively, or additionally, the time availability can comprise temporary non-availability, for example due to a postponement of a longer examination, an examination of a patient (for example an emergency patient) contrary to an (for example existing) appointment schedule. Alternatively, or additionally, temporary non-availability can comprise the medical device being out of operation, for example due to a defect and/or maintenance work. Maintenance work can also comprise an expansion and/or upgrade (for example of the software, in particular the special software) of the medical device.

The presence of trained personnel can enable more complex examinations. Alternatively, or additionally, the presence of trained staff, special software and/or special accessories can enable suitability for clinical trials. Further alternatively or additionally, the special software can assist the trained staff in scheduling the examination, performing the examination and/or in an operational sequence.

Suitability for clinical trials as a device parameter can relate to one or more types of examination.

The step of receiving the utilization request can comprise an input to a user interface and/or a query to a database system. The query to the database system can comprise querying one or more patient parameters and/or the patient's history.

The input to the user interface can comprise an input to a computer, tablet, smartphone and/or another user device.

The database system can comprise a hospital information system (HIS). The class of the totality of IT information-processing systems for acquiring, processing or and/or forwarding medical and/or administrative data in a hospital can be referred to as a HIS.

Alternatively, or additionally, the database system can comprise a radiology information system (RIS). The class of the totality of IT information-processing systems for acquiring, processing and/or forwarding medical and/or administrative data in a radiology department can be referred to as an RIS.

A radiology department can comprise a plurality of different types of imaging medical devices, for example MRT, CT, X-ray devices, ultrasound devices and/or PET.

The step of assigning one of the at least one medical device determined to be suitable to the received utilization request can comprise optimizing a set of assignments of different patients comprising the patient to medical devices from the group of medical devices.

The step of the optimizing can comprise the weighting of patient parameters and/or device parameters. Alternatively, or additionally, the assignment can be described by a translation matrix, for example for translation from patient parameters to device parameters.

The assignment and/or optimization of the set of assignments can be configurable. For example, a configuration can be changed if a medical device from the group of medical devices is restricted (or removed) and/or expanded and/or a new medical device is added. Alternatively, or additionally, a change to the configuration can comprise a change to the temporal availability and/or the average duration of the type of examination.

Optimization can comprise minimizing a linear combination of dimensionless cost functions. A dimensionless cost function can be assigned to each of the at least one patient parameter.

The cost function can comprise an (in particular normalized) function defining the distance from an ideal assignment of the respective patient parameters to one or more device parameters and/or to a medical device.

The linear combination of the dimensionless cost functions can comprise a weighted sum of the dimensionless cost functions.

The weighting can, for example, be used to class the urgency and/or location for a trauma patient as more important than technical specifications, for example a magnetic field strength of an MRT.

Alternatively, or additionally, higher magnetic field strengths can be classed as more important or given a higher weighting (also: weighted higher) for specific anatomical regions, for example smaller joints. This advantageously enables a higher resolution to be achieved, for example by selecting an appropriate medical device.

Alternatively, or additionally, lower radiation exposure can be desirable and/or legally required for predetermined patient groups, for example children and/or pregnant women. For example, an X-ray device with a lower radiation dose due to its construction may be weighted higher for the predetermined patient group.

Weighting can be selected differently for other, for example heavily normalized and/or less demanding (in particular routine) examinations. For example, energy saving, uniform utilization of the medical devices and/or the reservation of high-value (in particular research) devices by can be favored and/or achieved by means of appropriate weighting.

Optimization can comprise one or more steps of pairwise swapping of assignments of a patient to a medical device.

Pairwise swapping can correspond to an iteration of the optimization. Pairwise swapping can, for example, be continued until no better assignment is found. Alternatively, or additionally, pairwise swapping can be ended after a predetermined time.

The achievement of the object was described above with reference to the method. Features, advantages or alternative embodiments can also be transferred to the other aspects and vice versa. In other words, aspects (which are, for example, directed at agent apparatuses, a system or a computer program product) can be developed with the features described in connection with the method. Herein, the appropriate functional features of the method are embodied by appropriate substantive modules, in particular hardware modules or microprocessor modules, of the agent apparatus, the system or the product and vice versa.

According to a first apparatus aspect, a scheduler agent apparatus for assigning a medical device from a group of medical devices to an examination of a patient is provided. The scheduler agent apparatus comprises a receiver (Rx), which is embodied to receive a utilization request for examining a patient by means of a medical device from the group of medical devices. The utilization request is received from a patient agent apparatus. The utilization request comprises at least one patient parameter of the patient.

The scheduler agent apparatus further comprises a determining unit (determiner), which is embodied to determine the suitability of at least one medical device from the group of medical devices for the examination based on the at least one received patient parameter and at least one device parameter of the medical device. The scheduler agent apparatus further comprises an assigning unit (assigner), which is embodied to assign one of the at least one medical device determined to be suitable to the received utilization request for the examination of the patient based on the at least one received patient parameter and the at least one device parameter. The scheduler agent apparatus further comprises a first transmitter (Tx), which is embodied to transmit a notification comprising the assigned medical device determined to be suitable for the examination of the patient to the patient agent apparatus. The scheduler agent apparatus further comprises a second transmitter, which is embodied to transmit a further notification to a device agent apparatus associated with the assigned medical device. The further notification comprises the assigned examination of the patient.

The scheduler agent apparatus can comprise any feature described in connection with the method.

The scheduler agent apparatus can alternatively or additionally be embodied to execute the steps of the apparatus aspect.

According to a second apparatus aspect, a device agent apparatus for assigning a medical device from a group of medical devices to an examination of a patient is provided. The device agent apparatus is assigned to the medical device. The device agent apparatus comprises a first receiver, which is embodied to receive a notification comprising the assigned examination of the patient. The notification is received from a scheduler agent apparatus.

The device agent apparatus can further comprise a second receiver, which is embodied to receive an availability request relating to the medical device from the scheduler agent apparatus. Alternatively, or additionally, the device agent apparatus can comprise a transmitter, which is embodied to transmit availability information relating to the medical device to the scheduler agent apparatus.

According to a system aspect, a system for assigning a medical device from a group of medical devices to an examination of a patient is provided. The system comprises a scheduler agent apparatus according to the first apparatus aspect, a plurality of device agent apparatuses according to the second apparatus aspect, wherein each device agent apparatus is assigned to a medical device, and at least one patient agent apparatus for assigning a medical device from a group of medical devices to an examination of a patient. The patient agent apparatus comprises a receiver, which is embodied to receive at least one patient parameter for examining a patient. The patient agent apparatus further comprises a transmitter, which is embodied to transmit a utilization request for the examination of the patient by means of a medical device from the group of medical devices. The utilization request is transmitted to the scheduler agent apparatus. The utilization request comprises the at least one patient parameter of the patient.

According to a further aspect, a computer program product is provided. The computer program product comprises instructions, which, when the program is executed by a computer, cause the computer to execute the method according to the method aspect.

According to a further aspect, a computer-readable storage medium is provided. The computer-readable storage medium comprises instructions, which, when executed by at least one computer, cause the computer to execute the steps of the method according to the method aspect.

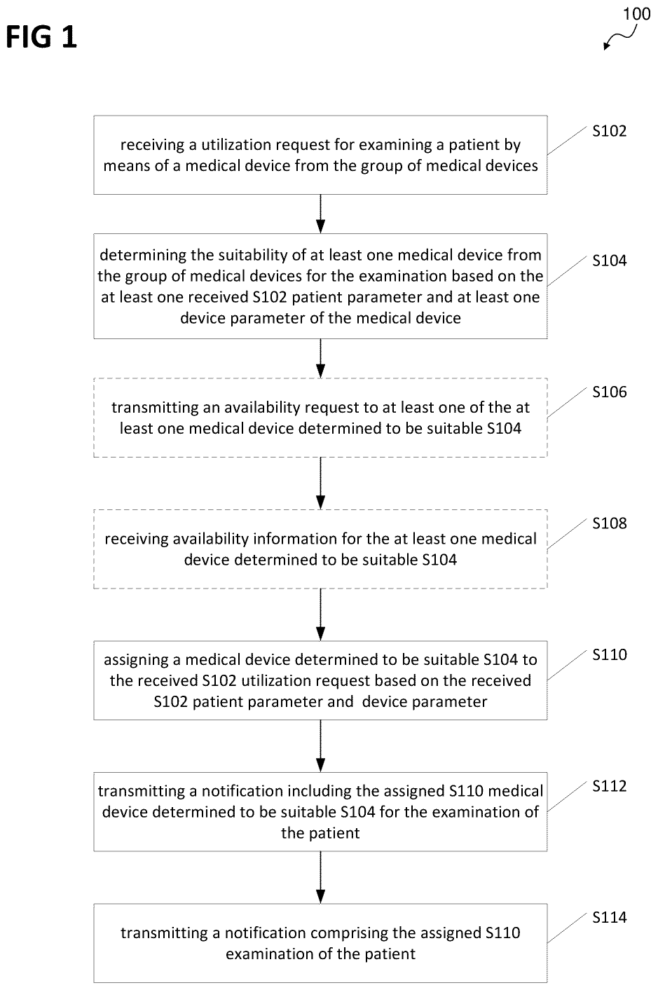

FIG. 1 is a schematic view of an exemplary embodiment of a computer-implemented method for assigning a medical device from a group of medical devices to an examination of a patient. The method is generally denoted by reference character 100. The method can be implemented in a scheduler agent apparatus.

The method 100 comprises a step S102 of receiving a utilization request for examining a patient by means of a medical device from the group of medical devices. The utilization request is received from a patient agent apparatus and comprises at least one patient parameter of the patient.

The method 100 further comprises a step S104 of determining the suitability of at least one medical device from the group of medical devices for the examination based on the at least one received S102 patient parameter and at least one device parameter of the medical device.

The method 100 further comprises a step S110 of assigning one of the at least one medical device determined to be suitable S104 to the received S102 utilization request for the examination of the patient based on the at least one received S102 patient parameter and the at least one device parameter.

The method 100 further comprises a step S112 of transmitting a notification comprising the assigned S110 medical device determined to be suitable S104 for the examination of the patient. In step S112, the notification is transmitted to the patient agent apparatus.

The method 100 further comprises a step S114 of transmitting a notification comprising the assigned S110 examination of the patient. In step S114, the notification is transmitted to a device agent apparatus associated with the assigned S110 medical device.

The method 100 optionally further comprises a step S106 of transmitting an availability request to at least one of the at least one medical device determined to be suitable S104. The availability request can be transmitted to the device agent apparatus associated with the respective medical device determined to be suitable.

The method 100 furthermore optionally comprises a step S108, S108′ of receiving availability information for the at least one medical device determined to be suitable S104. The availability information can be received in response to the availability request transmitted in the step S106. The availability information can be received from the device agent apparatus associated with the respective medical device determined to be suitable.

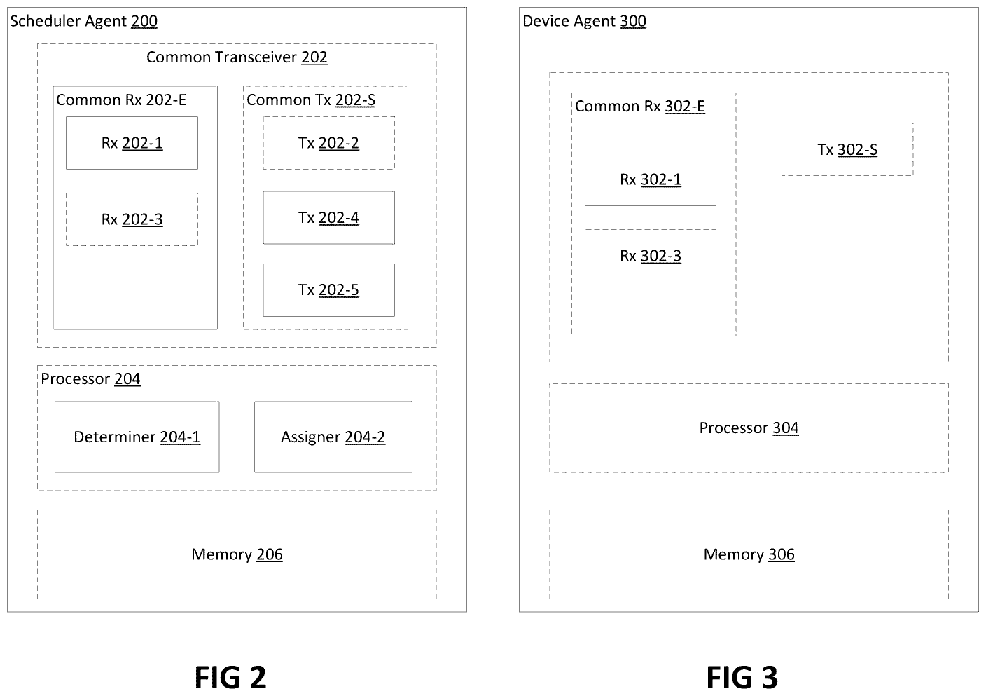

FIG. 2 is a schematic view of an exemplary embodiment of a scheduler agent apparatus for assigning a medical device from a group of medical devices to an examination of a patient. The scheduler agent apparatus is generally denoted by reference character 200.

The scheduler agent apparatus 200 comprises a receiver 202-1, which is embodied to receive a utilization request for examining a patient by means of a medical device from the group of medical devices. The utilization request can be received from a patient agent apparatus and comprise at least one patient parameter of the patient.

The scheduler agent apparatus 200 further comprises a determining unit (determiner) 204-1, which is embodied to determine the suitability of at least one medical device from the group of medical devices for the examination based on the at least one received patient parameter and the at least one device parameter of the medical device.

The scheduler agent apparatus 200 further comprises an assigning unit (assigner) 204-2, which is embodied to assign one of the at least one medical device determined to be suitable to the received utilization request for the examination of the patient based on the at least one received patient parameter and the at least one device parameter.

The scheduler agent apparatus 200 further comprises a first transmitter 202-4, which is embodied to transmit a notification comprising the assigned medical device determined to be suitable for the examination of the patient. The first transmitter 202-4 is embodied to transmit the notification to the patient agent apparatus.

The scheduler agent apparatus 200 further comprises a second transmitter 202-5, which is embodied to transmit a notification comprising the assigned examination of the patient. The transmitter 202-5 is embodied to transmit the notification to a device agent apparatus associated with the assigned medical device.

The scheduler agent apparatus 200 optionally further comprises a further transmitter 202-2, which is embodied to transmit an availability request to at least one of the at least one medical devices determined to be suitable. The availability request can be transmitted to the device agent apparatus associated with the respective medical device determined to be suitable.

The scheduler agent apparatus 200 furthermore optionally comprises a further receiver 202-3, which is embodied to receive availability information for the at least one medical device determined to be suitable. The availability information can be received in response to the transmitted availability request. The availability information can be received from the device agent apparatus associated with the respective medical device determined to be suitable.

The transmitters 202-4 and 202-5, and optionally the further transmitter 202-2, can be combined in a common transmitter 202-S. Alternatively or additionally, the receiver 202-1 and the optional further receiver 202-3 can be combined in a common receiver 202-E. Further alternatively or additionally, all transmitters 202-4, 202-5 (and optionally 202-2) and receivers 202-1 (and optionally 202-3) can be combined in a common transmitting/receiver (transceiver) 202. The common transmitting/receiver (transceiver) 202 can also be referred to as a data interface.

Further, the determining unit 204-1 and the assigning unit 204-2 of the scheduler agent apparatus 200 are optionally combined in a common unit 204, for example a processor.

The scheduler agent apparatus 200 optionally further comprises a memory unit 206.

FIG. 3 is a schematic view of an exemplary embodiment of a device agent apparatus for assigning a medical device from a group of medical devices to an examination of a patient. The device agent apparatus is generally denoted by reference character 300. The device agent apparatus 300 is assigned to the medical device.

The device agent apparatus 300 comprises a first receiver 302-1, which is embodied to receive a notification comprising the assigned examination of the patient. The device agent apparatus 300 is embodied to receive the notification from a scheduler agent apparatus, for example the apparatus 200.

The device agent apparatus 300 optionally comprises a second receiver 302-3, which is embodied to receive an availability request relating to the medical device from the scheduler agent apparatus, for example the apparatus 200. The second receiver 302-3 can be embodied to receive the availability request from the scheduler agent apparatus, for example the apparatus 200.

The device agent apparatus 300 furthermore optionally comprises a transmitter 302-S, which is embodied to transmit availability information relating to the medical device to the scheduler agent apparatus. The transmitter 302-S can be embodied to transmit the availability information to the scheduler agent apparatus, for example the apparatus 200.

The receivers 302-1 and 302-3 can be combined in a common receiver 302-E. Alternatively or additionally, the receivers 302-1, 302-3 and the transmitter 302-S can be combined in a common transmitting/receiver 302. The common transmitting/receiver 302 can also be referred to as a data interface.

The device agent apparatus 300 optionally further comprises a processor 304 and/or a memory unit 306.

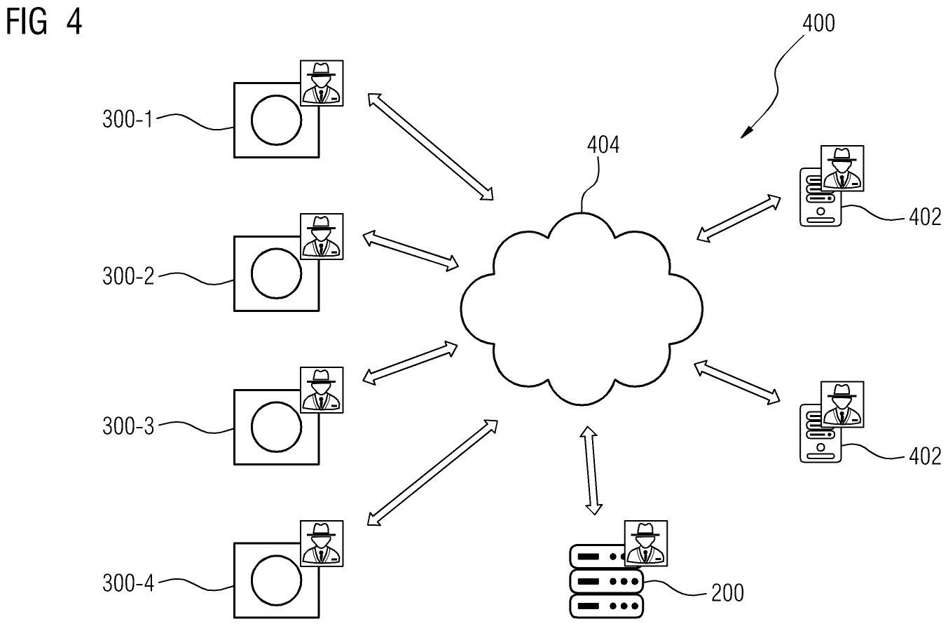

FIG. 4 shows an exemplary embodiment of a system 400. The system 400 comprises a scheduler agent apparatus 200 and a plurality of device agent apparatuses 300.

In the exemplary embodiment in FIG. 4, the first device agent apparatus 300-1 can, for example, be assigned to an MRT, the second device agent apparatus 300-2 can likewise be assigned to an MRT, the third device agent apparatus 300-3 can be assigned to a CT and the fourth device agent apparatus 300-4 can be assigned to a further imaging device, for example an X-ray device.

By way of example, the system 400 in FIG. 4 further comprises two patient agent apparatuses 402 for assigning a medical device from a group of medical devices to an examination of a patient.

Each patient agent apparatus 402 can comprise a receiver, which is embodied to receive at least one patient parameter for examining a patient.

Each patient agent apparatus 402 can further comprise a transmitter, which is embodied to transmit, to the scheduler agent apparatus 200, a utilization request for the examination of the patient by means of a medical device from the group of medical devices. The utilization request can comprise the at least one patient parameter of the patient.

The numbers of device agent apparatuses 300 and patient agent apparatuses 402 in FIG. 4 are in each case shown by way of example. Other numbers of respective types of apparatus that are independent of one another are possible. For example, a radiology department can comprise a plurality of medical devices and assigned device agent apparatuses 300. Alternatively, or additionally, a radiology department can, for example, comprise one (or a few) centralized patient agent apparatuses 402. Further alternatively or additionally, the scheduler agent apparatus 200 can be in data communication with a plurality of radiology departments, for example within a city, for example via a cloud-based infrastructure 404 as depicted schematically in FIG. 4.

In the exemplary embodiment in FIG. 4, the object of directing a specific patient to a specific medical device (for example an MRT scanner) is delegated to a (for example cloud-based) assignment system (also: negotiation system) 400 that determines the most appropriate medical device (in particular an imaging medical device, scanner for short) for the individual diagnostic imaging requirement. The determination of the most suitable medical device involves analyzing the required device (also: scanner) capacities on the basis of predetermined (and/or important) factors (also: criteria).

A predetermined (and/or important) factor can comprise the type of imaging requirement. For example, an orthopedic examination can require specific equipment, for example MRT coils (also: antenna coils), and/or special software (for example scanner software) and/or licenses (in particular software licenses). Alternatively, or additionally, a high field strength (for example magnetic field strength) can be preferred for optimum image quality.

Alternatively, or additionally, a predetermined (and/or important) factor can comprise a patient's participation in a research study. For example, a patient who has agreed to participate in a research study can be directed to another medical device (also: scanner) (for example other than that intended for a conventional routine examination), where additional research pulse sequences are available.

Further alternatively or additionally, a predetermined (and/or important) factor can comprise a specific patient condition. For example, if a patient has metal implants and/or a pacemaker, is claustrophobic and/or is severely overweight, some medical devices (also: scanners) may be unsuitable and/or specific medical devices (also: scanners) may be preferable for examining such cases.

Further alternatively or additionally, a predetermined (and/or important) factor can comprise guidelines for the facility's selection of medical devices (also: scanners). For example, routine examinations with medical devices (also: scanners) which are provided with conventional equipment, can be performed with a high throughput.

Further alternatively or additionally, a predetermined (and/or important) factor can comprise availability and/or location. For example, an emergency patient often has to be examined (also: scanned) within two hours and/or cannot be transported to another building.

Each criterion (also: factor) can have a different priority. Alternatively, or additionally, a combination of preferred factors (and/or capabilities of the medical device) can be specified, for example by the patient agent apparatus 402. In the exemplary embodiment shown in FIG. 4, the task of directing a specific patient to a specific medical device (for example to an MRT scanner) is delegated to cloud-based processing that determines the most suitable medical device (for example scanner) for the individual diagnostic imaging.

According to the disclosure, two main modules can be implemented. A first main module comprises an algorithm that translates input variables (for example patient imaging requirement with constraints and/or institutional guidelines) into a list of requirements for the capabilities of the medical device (also: scanner capabilities) for individual cases. A second main module can execute a cloud-based activity that identifies a list of medical devices (also: scanners) that meet the requirements for each individual case. The one or more medical devices (also: scanners) can be identified through the use of the needs-based assignment system 400 (which can also be referred to as a market-based negotiation system) with software agents 200, 300 and 402.

The list resulting from the second main module, in particular identified medical devices (also: scanners), can be presented directly to a user (for example a member of the hospital staff) as input for the patient scheduling process. Alternatively, or additionally, the list resulting from the second main module can be integrated into patient scheduling software.

In particular, according to the disclosure, the second main module can be implemented by an agent-based assignment mechanism (also: negotiation mechanism).

The creation of an optimal, or near optimal, assignment (for example comprising an appointment schedule) for patients considering criteria (also: factors), for example the best suitability of a type of medical devices (also: scanner type), availability and suitable location, is conventionally a non-trivial and highly dynamic task.

According to the disclosure, requirements for the assignments (for example comprising appointment schedules) are met by using software agents 200, 300 and 402 with suitable communication strategies (which can also be referred to as negotiation scenarios).

Software agents 200, 300 and 402 can comprise independent software components that are each able to follow their own goals. They can exchange messages as part of communication protocols (also: negotiation protocols or negotiation scenarios) in order to pursue their goals.

According to the disclosure, an open needs-based communication platform (which can also be referred to as a market-based negotiation platform) can be created with the following actors, which are each represented by an agent (for example an agent comprising software).

A device agent apparatus 300 (also: device agent or scanner agent for short) is assigned to or represents a medical device (also: scanner). The, or each, device agent apparatus 300 has the appropriate information, for example the type of medical device (also: scanner type), location, diagnostic capabilities, and/or limitations (for example with regard to the software provided, special accessories and/or time availability).

A patient agent apparatus 402 (also: patient agent for short) represents the interests of the patient in order to meet the requirements based on a series of patient parameters, for example the type of examination (also: scan), urgency and/or mobility.

A scheduler agent apparatus 200 (also: scheduler agent for short) is responsible for creating an assignment (for example as assignment comprising a schedule) based on the interests of the user (for example hospitals).

The interests of the user can correspond to and/or comprise the criteria (also: factors). For example, interests can comprise a schedule with minimal patient delays, maximum utilization of the medical devices (also: scanner utilization), minimal patient movement requirements, including scheduled downtimes for medical devices (also: scanners) for maintenance and/or the ability to respond to unforeseen events and/or disruptions. The unforeseen events and/or disruptions can, for example, comprise disruptions in the scheduled workflow due to large-scale emergency responses (for example major traffic accidents, in particular on the highway) or disruptions at the hospital (for example flood and/or fire).

The scheduler agent apparatus 402, a plurality of device agent apparatuses 300 and a plurality of patient agent apparatuses 402 are shown by way of example in FIG. 4. In the exemplary embodiment shown in FIG. 4, the scheduler agent apparatus 402 communicates with the device agent apparatuses 300 and with the patient agent apparatuses 402 by means of a cloud-based infrastructure 404.

Each unit—for example (in particular imaging) medical device, scheduler, patient—can be represented by an agent apparatus (in particular an agent apparatus comprising software) (also: software agents for short) 200; 300; 402.

The agent apparatuses 200; 300; 402 each pursue their own goals by negotiating and/or exchanging messages with other agent apparatuses 200; 300; 402. The role of the device agent apparatuses 300; 300-1; 300-2; 300-3; 300-4 (also: scanner agents for short), as shown in FIG. 4, for example, can consist in and/or comprise managing the relevant information about the medical device (also: scanner), for example its capabilities (for example comprising a magnetic field strength of an MRT) and/or its location.

The patient agent apparatuses 402 (also; patient agents for short) can pursue the goal of accommodating the patient as quickly and comfortably as possible.

The scheduler agent apparatus 200 (also: scheduler agent for short) can attempt to create assignments (for example including schedules) in order to meet the goals of the patient agent apparatuses 402 while simultaneously maximizing the utilization of the medical devices (also: scanners) (for example for the entire group and/or fleet of medical devices of one type or kind). In addition, the scheduler agent apparatus 200 can take care of the dynamic elements of the system 400 and respond to events, in particular unforeseen events, (for example immediately and/or promptly).

The communication paths (also: communication scenarios or negotiation scenarios) between the agent apparatuses 200; 300; 402 can be drawn up and/or defined in advance. The communication paths (also: negotiation scenarios) can include the type and/or content of the messages exchanged.

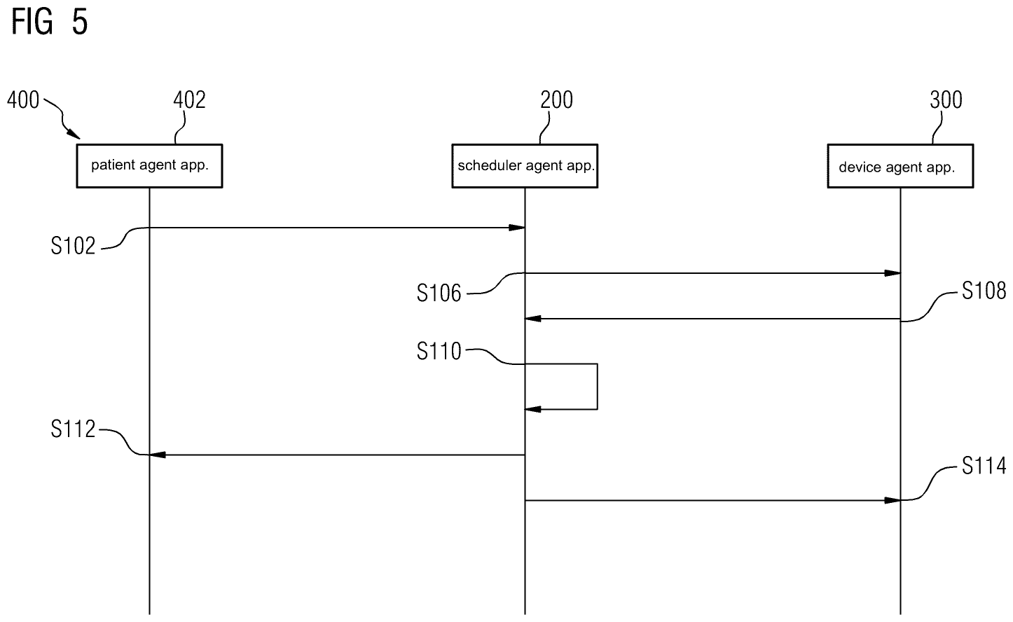

A communication scenario can represent the patient's request for an assignment (for example comprising an appointment schedule) based on specified criteria (also: factors) as shown in FIG. 5.

In FIG. 5, at reference character S102, a patient agent apparatus 402 transmits a utilization request (which can also be called an assignment request and/or, for example, comprising an appointment request) based on specified patient parameters (also: patient information) to the scheduler agent apparatus 200. At reference character S106, the scheduler agent apparatus 200 queries the current availability of medical devices from the respective assigned device agent apparatuses 300 (for example from all medical devices and/or scanners) and, at reference character S108, receives availability information from each of the queried device agent apparatuses 300 (for example from all device agent apparatuses 300 and thus information about the availability of all medical devices in the group and/or fleet).

At reference character S110, the scheduler agent apparatus 200 then creates a new assignment (also: assignment schedule, for example comprising an appointment schedule) for example based on the availabilities of the medical devices, the respective device type (also: scanner type), for example according to the request from the patient agent apparatus 402, and/or the distance to the nearest medical device (also: scanner). When the assignment (for example comprising the schedule) has been created, it is forwarded to the respective (or all) patient agent apparatus 402 at reference character S112 and to the device agent apparatus 300 at reference character S114.

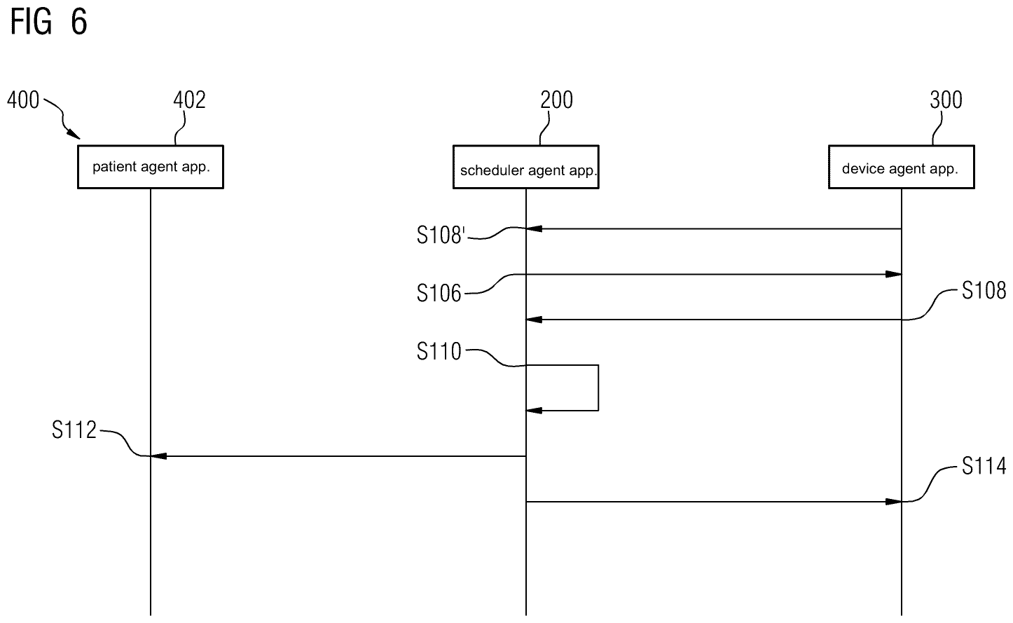

A further type of communication scenario comprises unforeseen events relating, for example, to a medical device (in particular an imaging medical device) (also: scanner). The scheduler agent apparatus 200 has to respond immediately in order to reschedule the current assignment (comprising the current schedule), as shown in FIG. 6.

After receiving information about a scheduled event, for example regular maintenance, and/or an unforeseen event, for example component failure and/or flooding, from the device agent apparatus 300 at reference character S108′, at reference character S106, the scheduler agent apparatus 200 queries, for example, all available time windows for all medical devices (also: scanners) (for example for all medical devices of a device type) and, at reference character S108, receives the respective availability information.

Similarly to the exemplary embodiment shown in FIG. 5, for example, the scheduler agent apparatus 200 creates a new assignment (also: new schedule) and notifies this to all parties involved, for example to the one or more patient agent apparatuses 402 at reference character S112 and the one or more device agent apparatuses 300 at reference character S114.

The creation of assignments (for example comprising schedules) is an aspect according to the disclosure. The scheduler agent apparatus 200 creates an assignment (for example comprising a schedule) that attempts to meet a series of criteria (also: factors) specified by the patient agent apparatuses 402 and device agent apparatuses 300. The criteria (also: factors) can, for example, comprise the type of examination (also: scan) required for a specific patient, the distance between the patient and the medical device, the desired time window for the examination (also: scan), the degree of utilization of a specific medical device (also: scanner), maintenance requirements and/or the treatment of unforeseen events such as fire, flood and/or component failure.

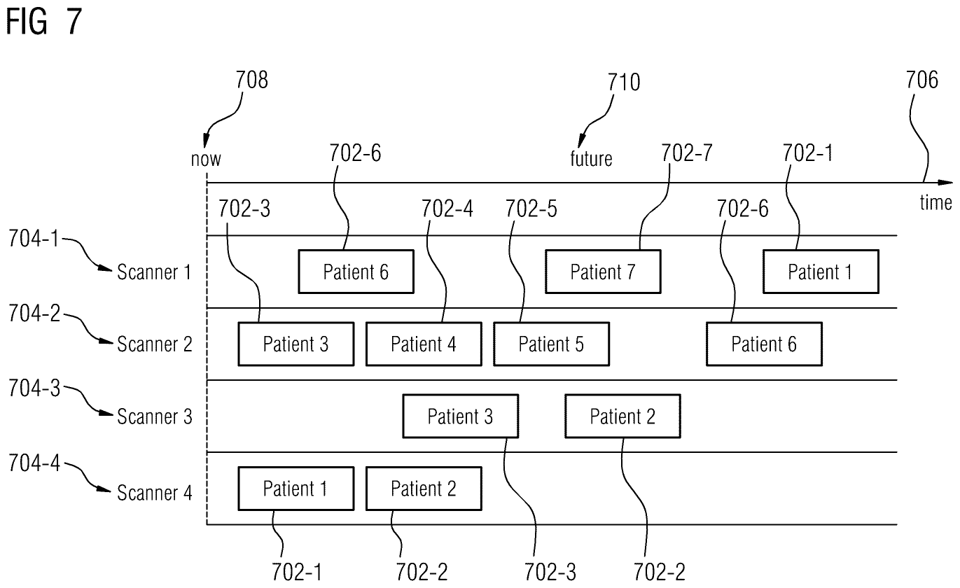

FIG. 7 shows by way of example an assignment (for example comprising a schedule). In the exemplary embodiment in FIG. 7, four medical devices (“scanners”) 704-1; 704-2; 704-3; 704-4 are listed to which patients 702-1; 702-2; 702-3; 702-4; 702-5; 702-6; 702-7 are assigned along a time axis 706. Each medical device 704-X (X=1, 2, 3, 4) can be assigned to a device agent apparatus 300-X. Alternatively or additionally, the patients 702-X (X=1, 2, 3, 4, 5, 6, 7) can be represented by one or more patient agent apparatuses 402.

The exemplary embodiment shown in FIG. 7 for a schedule which is the result of communication between a plurality of device agent apparatuses 300, at least one patient agent apparatus 402 and one scheduler agent apparatus 200 shows time windows from a present time 708 to a time in the future 710, wherein a series of required parameters (in particular patient and/or device parameters) are allocated. Examples of parameters comprise the type of medical device (also: scanner) required for a specific patient, availability of the medical device (also: scanner) and/or the location thereof.

The scheduler agent apparatus 200 can attempt to allocate an appointment in the future that ideally meets the requirements. However, due to limited and sometimes unforeseen events, this will not always be possible.

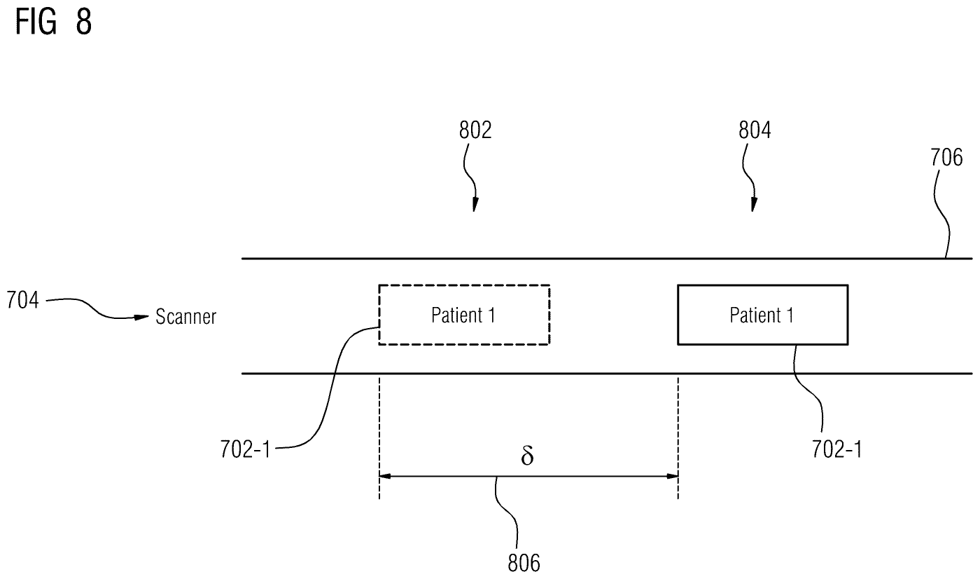

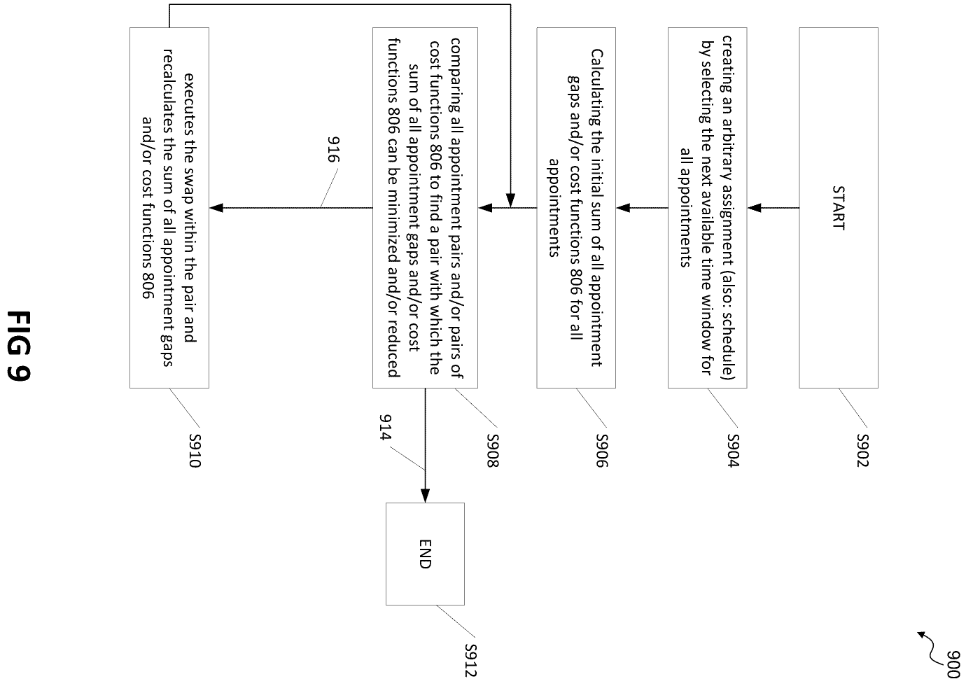

An important exemplary factor in creating an assignment (for example comprising a schedule) is the “appointment gap” δ shown in FIG. 8 at reference character 806 between the ideal position 802 of the time window and its (for example actual) scheduled position 804.

The exemplary “appointment gap” δ at reference character 806 comprises a single value (and/or parameter) of a linear combination of all parameters required for the schedule.

The required parameters can also be referred to as cost functions. Each cost function can be normalized with respect to a value relevant for the corresponding parameter. Alternatively, or additionally, each cost function can be dimensionless.

Further alternatively or additionally, the individual value can correspond to an overall cost function. For example, the distance between the patient and the medical device (also: scanner) can have a negative impact on the “appointment gap” 806. An earlier appointment in the future can have a positive effect, as can better image quality of the available medical device (also: scanner).

The scheduler agent apparatus 200 can initially create an arbitrary assignment (for example comprising a schedule). For example, not all appointments are scheduled at the ideal time.

The scheduler agent apparatus 200 can attempt to swap some of the appointments in order to minimize the appointment gap δ (and/or the cost functions for a plurality of parameters, and/or the overall cost function) and reference character 806 between ideal and scheduled appointments. Alternatively, or additionally, the scheduler agent apparatus 200 can attempt to minimize the sum of the appointment gaps (and/or the cost functions for a plurality of parameters and/or the overall cost function) 806 for all appointments by swapping appointments.

According to one exemplary embodiment, it is only possible to swap assignments and/or appointments that meet the same requirements, such as, for example, the same device type (also: scanner type).

FIG. 9 shows an exemplary embodiment of a scheduling algorithm 900 (also: optimization algorithm) that can be executed by the scheduler agent apparatus 200.

The exemplary method 900 in FIG. 9 begins at reference character S902.

At reference character S904, the scheduler agent apparatus 200 creates an arbitrary assignment (also: schedule) by selecting the next available time window for all appointments while considering requirements such as the appropriate device type (also: scanner type).

At reference character S906, the scheduler agent apparatus 200 calculates the initial sum of all appointment gaps and/or cost functions 806 for all appointments.

At reference character S908, the scheduler agent apparatus 200 compares all appointment pairs and/or pairs of cost functions 806 in an attempt to find a pair with which the sum of all appointment gaps and/or cost functions 806 can be minimized (and/or reduced to the maximum degree).

If such a pair is found at reference character 916, the method is continued at reference character S910. At reference character S910, the scheduler agent apparatus 200 executes the swap within the pair and recalculates the sum of all appointment gaps and/or cost functions 806. Then, step S908 is performed again to search for further pairs.

If no reduction in the sum of all appointment gaps and/or cost functions 806 is found at reference character 914, the method ends at reference character S912. The new assignment and/or the new schedule are notified to the affected patient agent apparatuses 402 and device agent apparatuses 300, for example according to steps S112 or S114.

In each exemplary embodiment, communication between image requirement (for example by a patient agent apparatus 402) and a group of medical devices (for example comprising one device agent apparatus 300 per medical device) can take place via a scheduler agent apparatus 200 as a communication interface (and/or negotiation interface). In particular, communication can be cloud-based.

The technique according to the disclosure enables device utilization (also: scanner utilization) to be optimized, in particular for groups of (for example, imaging) medical devices (also: scanner fleets) in larger facilities and/or with a plurality of medical devices. The technique according to the disclosure can enable good scalability, for example with the number of medical devices (also: scanners) and/or the number of locations (for example without major modifications when adding a medical device and/or a location).

In a first exemplary embodiment, the medical device can comprise an MRT (and/or comprise MRT modality).

For example, a planned and/or requested examination can comprise a knee examination for a patient with problems with an artificial knee joint.

For example, the following information can be transmitted to the patient agent: a request for a knee examination to clarify problems with an artificial joint replacement, and the patient's weight, for example 210 kg, and/or the information that the patient is severely obese. The information can, for example, be input via a special input mask. Alternatively, or additionally, there can advantageously be a connection to a hospital and/or radiology information system (HIS and/or RIS) in which data, in particular from previous examinations and/or treatments of the patient, are at least partially already available and/or queried.

For a radiology department at the hospital where an examination is to take place, an algorithm can be implemented by means of which the scheduler agent apparatus 200 translates the requirement of the patient agent apparatus 402 into requirements for a suitable device (also: scanner). The algorithm can relate patient parameters to the device parameters (also: scanner parameters), for example via relative weightings (also: weights). For example, a weighting for the relationship of patient body weight to the device field strength (also: scanner field strength) can have the value 0% so that the body weight is not relevant for the selection of the field strength. Alternatively, or additionally, a weighting of 100% can mean that it is mandatory for the parameter relationship to be observed, for example a patient parameter in a requirement for a contrast medium examination may make it necessary for the medical device (also: scanner) to have a contrast medium injection device. Values between 0% and 100% can mean that the parameter relationship is advantageous, but not mandatory, and should be matched as closely as possible when it is necessary to weigh up which patient ‘fits’ better to one or another medical device (also: scanner).

The relationships between patient parameters and device parameters can be summarized in a translation matrix. For example, the entries in the translation matrix can comprise the weightings.

The translation matrix for translating patient parameters to device parameters (also: scanner parameters) is preferably configurable, since it may depend on the preferences of the respective institutions (for example hospital and/or radiology practice) or changes can also be necessary from time to time, for example if there is a change in the group of medical devices and/or in guidelines for examinations by means of the medical devices.

In the example of a patient who, for example, has an artificial joint replacement and is overweight, the following requirements for the medical device (also: scanner) may arise, for example: measurement protocols for the body region ‘knee’ must be available (mandatory requirement for a knee examination); equipment with a local coil for the knee must be available (mandatory requirement for a knee examination by means of MRT); the system must have a tunnel diameter of at least 70 cm due to obesity (mandatory requirement); the patient bench must be suitable for 210 kg (mandatory requirement); field strength of 1.5 T (preferred requirement, for example with 80% weighting); system must be equipped with special examination protocols for implants (mandatory requirement in the case of artificial joint replacements); operating staff must be trained in examination methods and precautionary measures for patients with implants (mandatory requirement in the case of artificial joint replacements).

The scheduler agent apparatus 200 can match and/or negotiate these requirements with the device agent apparatuses 300 in order to identify a suitable medical device (also: scanner) and, in the case of a plurality of patients (for example with requirements that deviate from the detailed example of a patient given above), to achieve the optimum.

In a further exemplary embodiment, a medical device (also: scanner) can report a failure to the scheduler agent apparatus 200. The report can be made by means of the device agent apparatus 300 assigned to the medical device.

For example, the medical device (also: scanner) can be defective causing the device agent apparatus 300 to report to the scheduler agent apparatus 200 that the patients scheduled for the next day and/or the next 24 hours cannot be examined.

The scheduler agent apparatus 200 can become active and search for new assignments and/or appointments for the patients in question.

Alternatively, or additionally, a patient can urgently require a new assignment and/or a new appointment at short notice (for example due to an accident) and, for this purpose, the assignment and/or the appointment of another less urgent patient (for example for a routine checkup) may be delayed.

The following table summarizes exemplary patient parameters that can be stored in and/or entered into a patient agent apparatus 402. The last column lists requirements for device parameters (also: technical requirements and/or technical parameters), in particular for MRT, by way of example depending on the respective patient parameter.

Patient parameters, for example stored in a patient
agent apparatus, and suitability of medical devices

| | Typical values | |
|---|---|---|
| Parameter | (selection/example) | Relevance |
| Region to be examined | Skull/heart/abdomen/hip/ | Scanner must support |
| | knee/ankle and/or DRG | examinations in this body |
| | code/ICD-10 code/CPT code | region (for example by |
| | | appropriate measuring |
| | | methods and evaluation |
| | | software, suitable MR coils, etc.) |
| Examination | T1w/T2w/PD-FS/ | Scanner must have |
| parameters/ | angiography/contrast | appropriate equipment |
| requirement regarding | medium administration and/or | (dedicated measuring |
| diagnostic image | CPT code | sequences, evaluation |
| contrasts/imaging types | | software, contrast medium |
| | | injector, appropriately trained |
| | | staff, etc.) |
| Patient weight | 10-200 kg | In particular in the case of very |
| | | heavy or obese patients there |
| | | are special requirements for |
| | | the scanner (weight restriction |
| | | for patient benches, |
| | | examination tube diameter) |
| Patient is | Yes/no | |
| claustrophobic | | |
| Patient is able to lie | Yes/no | |
| still for several | | |
| minutes | | |
| Patient is able to hold | Yes/no | If no, a scanner with special |
| breath for at least 15 s | | equipment that allows |
| | | measurement with free |
| | | breathing is required |
| Required field strength | 0.5 T, 1.5 T/3 T/7 T/not | All field strengths have |
| | relevant | advantages and disadvantages |
| | | for different measurements |
| | | that apply differently to |
| | | different measurements |
| Patient has metallic | Yes/no/type of implant | Lower field strengths are more |
| implant | | suitable for metallic implants |
| | | than higher field strengths. |
| | | Furthermore, metallic implants |
| | | cause significant disruptions of |
| | | the magnetic field, so that |
| | | special measuring methods are |
| | | required, which are not usually |
| | | available on every Scanner. |
| Examination as part of | Clinical trial as part of a | Scanner must have |
| a special study | research project/ | appropriate equipment |
| | development of new | (dedicated measuring |
| | diagnostic methods | sequences, evaluation |
| | | software, contrast medium |
| | | injector, appropriately trained |
| | | staff, etc.), special accessories, |
| | | a dedicated scanner is often |
| | | required to ensure |
| | | comparability of outcomes. |
| Timeframe for the | Within 12 h/14 days/in the | It is necessary to find a |
| examination | period August 13-September 1 | suitable scanner available in |
| | | this time period. |

The following table summarizes exemplary device parameters (also: scanner parameters) that can be stored in a device agent apparatus 300. In particular for MRT, the device parameters can comprise properties and/or capabilities of the medical device equipment, for example with special accessories, and/or availability.

Device parameters, for example stored in a device
agent apparatus, and suitability for examinations

| Parameter | Typical values | Relevance |
|---|---|---|
| Field strength | 0.5/1.5/3/7 tesla | Higher field strengths typically |
| | | enable higher image quality |
| | | (higher spatial resolution, less |
| | | image noise) |
| Diameter of the | 50/60/70/80 cm | Claustrophobia, examination of |
| patient bore | | obese patients |
| Max. patient | 200 kg/300 kg | Weight limit of the patient bench |
| weight | | |
| Local coils | Dedicated coils for head/neck/ | Optimal image quality virtually |
| | spine/abdomen/joints | always requires a local coil |
| | | (=signal receiver) adapted |
| | | to the body region. |
| Special | Injector for contrast medium, | Special accessories are often |
| accessories for | optical stimulator for fMRT, ECG | only available on individual |
| special | sensor, movable patient table, | devices (for example because |
| examinations | suitability for special interventions | they are tailored to a specific |
| | (organ puncture, tumor biopsy, etc.) | scanner type, high price, etc.) |
| Dedicated | Skull (standard) | Measuring methods differ |
| examination | spine (cervical spine, thoracic | greatly for different body |
| types | spine, lumbar spine) | regions and diagnostic issues −> |
| | Spine (multi-station) | the scanner must be suitable or |
| | Knee (standard, no implants) | equipped as appropriate for the |
| | Knee (implants) | examination in question |
| | Angiography (head, neck, | (hardware and software). |
| | peripheral), and/or CPT codes | |
| | and/or DRG or ICD- 10 categories | |
| | supported: for example, “CPT | |
| | 72159 - MR angio spine | |
| | w/o&w/dye” | |
| Dedicated | fMRT (functional brain | Some examination methods are |
| software | examination)/morphometry | tied to special software packets |
| licenses | package/implant imaging | that are only available on the |
| | methods | corresponding scanners via |
| | | license models. |
| Availability | Mo.-Fr. 7 a.m.-8 p.m. | Presence of qualified staff, |
| | | unavailability (for example |
| | | maintenance periods, use for |
| | | other purposes) |
| Current status | Free/delays with current | In practice, there are frequently |
| | examination/occupied by | unforeseen delays, vacancies |
| | emergency patients/unavailable | (patient no-show) or blockages |
| | due to defect | (emergency patient, system defect) |

According to a further exemplary embodiment, the medical device can comprise a CT.

When CT is used as the modality, the following patient parameters and/or device parameters can, for example, be considered in a scheduler agent apparatus 200 for the assignment (also: allocation) of the patient for specific examinations.

In addition to conventional single-source CTs, there are also dual-source CTs, i.e., computed tomography units with one or two X-ray sources. The use of two X-ray sources and detectors enable examination times to be reduced; this is particularly beneficial for trauma patients. Alternatively, or additionally, dual-Source CTs enable improved temporal resolution and are thus able to depict moving structures better than conventional single-source CTs. Improved temporal resolution is, for example, very advantageous in cardiac imaging and, for example, enables 4D images of the coronary arteries.

Technical progress has meant that the required X-ray dose has become increasingly lower. Depending upon the requirements of the examination, it is not always the lowest dose, and thus not always the latest device, that is required. However, due to the long-term effects of X-rays, a low X-ray dose is preferred for children and/or adolescents. Alternatively, or additionally, a low X-ray dose (for example as a single dose) is preferred for repeated exposure, for example if the outcome of therapy is monitored by CT.

Requirements for spatial and/or temporal resolution can also represent criteria for the selection of a CT device. For example, high-resolution computed tomography is preferred for lung disease. For example, high spatial and/or temporal resolution is required for images of the beating heart.

1.0 According to a Further Exemplary Embodiment, the Medical Device can Comprise PET.

In oncology, for example, PET (positron emission tomography, for example as a modality) provides valuable information about metabolic processes in tumor tissue that is not possible with other methods. PET devices can, for example, depict the metabolic rate of glucose in the body which can in particular be used to distinguish between benign and malignant tumors. For example, due to their increased glucose metabolism, malignant tumors (for example compared to benign tumors) take up more contrast medium that contains radiolabeled glucose with a short decay time and is administered intravenously to the patient before the examination. Alternatively, or additionally, the PET method can measure the radiation occurring in the body in a spatially resolved manner.

According to further exemplary embodiments, combinations of two or more (in particular imaging) medical devices (also: scanners) are possible.

A combination of PET with MRT or CT in a single device (for example referred to as a PET/MRT scanner or PET/CT scanner) enables the advantages of the respective modality and/or the respective method (for example PET: depiction of metabolic processes, but relatively low spatial resolution; MRT: high soft tissue contrast, but relatively long measurement times; and/or CT: high speed and high resolution, but low soft tissue contrast).

The selection (and/or assignment) of a medical device by the scheduler agent apparatus 200, for example based on one or more device parameters, can depend on the combination of device types (for example PET/MRT and/or PET/CT) and on patient parameters, as shown by way of example in the following. For example, in the presence of an implant, a PET/MRT combination is not usually suitable as a medical device, while a PET/CT combination PET/CT is usually possible.

Possible combinations of different device types and applications

| | Medical device type | |
|---|---|---|
| Parameter | (also: scanner type) | Comment |
| Patient with implant | PET/CT | Implant produces strong |
| | (−> PET/MRT not | image interference |
| | possible!) | |
| Ventilation or | PET/CT | Required devices not |
| sedation/anesthesia | | generally MRT compatible |
| required | | |
| Emergency patient | PET/CT | CT significantly faster than |
| (time-critical) | | MRT |
| Use of X-rays not | PET/MR | For example, any use of |
| justifiable | | X-rays should be avoided |
| | | with children, and thus |
| | | MRT is preferable |

Finally, reference is made to the fact that the descriptions of the disclosure and the exemplary embodiments are in principle not to be understood restrictively with respect to a specific physical implementation of the disclosure. All features explained and shown in connection with individual embodiments of the disclosure can be provided in different combinations in the subject matter according to the disclosure in order to simultaneously implement their advantageous effects.

The scope of protection of the present disclosure is given by the following claims and is not limited by the features explained in the description or shown in the figures.

It is in particular evident to a person skilled in the art that the disclosure can be used not only for the assignment of imaging medical devices to examinations of patients, but also for other medical devices used for time-limited examination and/or treatment of a patient. Furthermore, the components of each agent-apparatus (in particular the scheduler agent apparatus and the device agent apparatus) and/or the components of the system can be implemented distributed over a plurality of physical products.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A computer-implemented method for assigning a medical device from a group of medical devices to an examination of a patient, comprising:
receiving, from a patient agent apparatus, a utilization request for examining a patient using a medical device from the group of medical devices, wherein the utilization request comprises at least one patient parameter of the patient;
determining a suitability of at least one medical device from the group of medical devices for the examination based on the at least one received patient parameter and at least one device parameter of the medical device;
assigning one of the at least one medical device determined to be suitable to the received utilization request for the examination of the patient based on the at least one received patient parameter and the at least one device parameter;
transmitting, to the patient agent apparatus, a notification comprising the assigned medical device determined to be suitable for the examination of the patient; and
transmitting, to a device agent apparatus, associated with the assigned medical device, a notification comprising the assigned examination of the patient.

2. The method as claimed in claim 1, wherein the group of medical devices comprises a type of medical devices for a predetermined type of patient examinations.

3. The method as claimed in claim 1, wherein the medical device comprises an imaging device, and/or wherein the group of medical devices comprises a group of imaging devices.

4. The method as claimed in claim 1, wherein the imaging device, and/or the group of imaging devices, is:
a magnetic resonance tomography unit (MRT);
a computed tomography unit (CT);
an X-ray device;
an ultrasound device;
a positron emission tomography unit (PET); and/or
a combination of PET and CT, and/or a combination of PET and MRT.

5. The method as claimed in claim 1, further comprising:
transmitting an availability request to at least one of the at least one medical device determined to be suitable; and/or
receiving availability information for the at least one medical device determined to be suitable.

6. The method as claimed in claim 1, wherein the at least one patient parameter is:
a medical question;
a part of the patient's body to be examined;
a patient's physique, in particular height and/or weight;
a presence of an implant at or near the part of the patient's body to be examined;
a patient's age;
a patient's gender;
a patient's mobility;
a patient's mental fitness;
a patient's physical fitness;
an urgency of the examination of the patient;
a request for a device parameter; and/or
a participation in a clinical trial.

7. The method as claimed in claim 1, wherein the at least one device parameter is:
a location of the medical device;
a design of the medical device, in particular comprising a tube, semi-open and/or framework-based;
a loading capacity and/or mobility of a patient bench;
a presence of special accessories;
a field strength, in particular magnetic field strength;
a number of X-ray sources and associated detectors;
an image quality;
a presence of special software for performing and/or evaluating the examination;
an average duration of a type of examination corresponding to the utilization request;
a time availability;
a presence of trained personnel; and/or
a suitability for clinical trials.

8. The method as claimed in claim 1, wherein receiving a utilization request comprises an input to a user interface and/or a query to a database system.

9. The method as claimed in claim 1, wherein assigning one of the at least one medical device determined to be suitable to the received utilization request comprises optimizing a set of assignments of different patients comprising the patient to medical devices from the group of medical devices.

10. The method as claimed in claim 9, wherein the optimization comprises minimizing a linear combination of dimensionless cost functions, wherein a dimensionless cost function is assigned to each of the at least one patient parameter.

11. The method as claimed in claim 10, wherein the linear combination comprises a weighted sum of the dimensionless cost functions.

12. The method as claimed in claim 9, wherein the optimization comprises one or more steps of pairwise swapping of assignments of a patient to a medical device.

13. A non-transitory computer-readable storage medium with an executable program stored thereon, that when executed, instructs a processor to perform the method of claim 1.

14. A scheduler agent apparatus for assigning a medical device from a group of medical devices to an examination of a patient, comprising:
a receiver configured to receive, from a patient agent apparatus, a utilization request for examining a patient by a medical device, wherein the utilization request comprises at least one patient parameter of the patient;
a determiner configured to determine a suitability of at least one medical device from the group of medical devices for the examination based on the at least one received patient parameter and at least one device parameter of the medical device;
an assigner configured to assign one of the at least one medical device determined to be suitable to the received utilization request for the examination of the patient based on the at least one received patient parameter and the at least one device parameter;
a first transmitter configured to transmit, to the patient agent apparatus, a notification comprising the assigned medical device determined to be suitable for the examination of the patient; and
a second transmitter configured to transmit, to a device agent apparatus associated with the assigned medical device, a notification comprising the assigned examination of the patient.

15. A device agent apparatus for assigning a medical device from a group of medical devices to an examination of a patient, wherein the device agent apparatus is assigned to the medical device, the device agent comprising:
a first receiver configured to receive, from a scheduler agent apparatus, a notification comprising the assigned examination of the patient.

16. The device agent apparatus as claimed in claim 15, further comprising:
a second receiver configured to receive an availability request relating to the medical device from the scheduler agent apparatus; and/or
a transmitter configured to transmit availability information relating to the medical device to the scheduler agent apparatus.

17. A system for assigning a medical device from a group of medical devices to an examination of a patient, comprising:
a scheduler agent apparatus including:
a receiver configured to receive, from a patient agent apparatus, a utilization request for examining a patient by a medical device, wherein the utilization request comprises at least one patient parameter of the patient;
a determiner configured to determine a suitability of at least one medical device from the group of medical devices for the examination based on the at least one received patient parameter and at least one device parameter of the medical device;
an assigner configured to assign one of the at least one medical device determined to be suitable to the received utilization request for the examination of the patient based on the at least one received patient parameter and the at least one device parameter;
a first transmitter configured to transmit, to the patient agent apparatus, a notification comprising the assigned medical device determined to be suitable for the examination of the patient; and
a second transmitter configured to transmit, to a device agent apparatus associated with the assigned medical device, a notification comprising the assigned examination of the patient;
a plurality of device agent apparatuses as claimed in claim 15, wherein each device agent apparatus is assigned to a medical device; and
at least one patient agent apparatus configured to assign a medical device from a group of medical devices to an examination of a patient, comprising:
a receiver configured to receive at least one patient parameter for examining a patient;
a transmitter configured to transmit, to the scheduler agent apparatus, a utilization request for the examination of the patient by means of a medical device from the group of medical devices, wherein the utilization request comprises the at least one patient parameter of the patient.
